(19) United States
(12) Patent Application Publication
(10) Pub. No.: US 2025/0157777 A1
(43) Pub. Date: May 15, 2025

(54) Generating Ionizing Radiation Using Laser Light

(71) Applicant: Infinite Potential Laboratories LP, Waterloo (CA)

(72) Inventors: Steve MacLean, Sainte Catherine de Hatley (CA); Stéphane Payeur, Montréal (CA); Jeffrey Alan Powell, Ste-Julie (CA); Simon Carrier-Vallières, Lemoyne (CA); Sylvain Fourmaux, Drummondville (CA); François Légaré, St-Eustache (CA); François Fillion-Gourdeau, La Prairie (CA)

(73) Assignee: Infinite Potential Laboratories LP, Waterloo (CA)

(21) Appl. No.: 18/949,934

(22) Filed: Nov. 15, 2024

Related U.S. Application Data
(63) Continuation of application No. PCT/CA2023/050702, filed on May 19, 2023.
(60) Provisional application No. 63/344,434, filed on May 20, 2022.

(51) Int. Cl. H01J 27/24; H05H 1/46

(52) U.S. Cl. CPC H01J 27/24; H05H 1/46

(57) ABSTRACT

In a general aspect, ionizing radiation is generated using laser light. In some aspects, an apparatus for generating ionizing radiation includes a laser configured to generate a pulse of laser light and a gas medium that has an interaction region therein. The apparatus also includes an optical element that has a reflective surface. The reflective surface defines a focal point in the interaction region of the gas medium. The reflective surface is configured to receive the pulse of laser light and focus the pulse of laser light at the focal point. In many variations, the pulse of laser light is configured to generate a plasma in the interaction region when focused at the focal point by the reflective surface. In these variations, the gas medium is configured to emit an ionizing radiation from the interaction region in response to the plasma being generated therein. The ionizing radiation includes electron radiation.

### CROSS-REFERENCE TO RELATED APPLICATIONS

This application is a continuation of International Application No. PCT/CA2023/050702, filed May 19, 2023 and entitled “Generating Ionizing Radiation Using Laser Lights,” which claims priority to U.S. Prov. Pat. App. No. 63/344,434, filed May 20, 2022 and entitled “Generating Ionizing Radiation Using Laser Light”. The disclosure of the priority applications are hereby incorporated by reference in its entirety.

### BACKGROUND

The following description relates to generating ionizing radiation using laser light.

Ionizing radiation is a form of energy capable of energizing electrons in the atoms and molecules of a material. In some cases, the energy of ionizing radiation is sufficiently high that the electrons may be ejected from the atoms and molecules. Examples of ionizing radiation include subatomic and electromagnetic radiation. Subatomic radiation may include protons, neutrons, electrons (e.g., beta rays), atomic nuclei (e.g., alpha particles), and nuclear fragments. Electromagnetic radiation may include X-ray and gamma-ray photons. Ionizing radiation can be generated by decaying radionuclides (e.g., ²³⁸U) or by devices specifically designed to do so (e.g., an X-ray tube).

### DESCRIPTION OF DRAWINGS

FIG. 1 is a schematic diagram, in cross section, of an example optical element that includes a high numerical parabolic reflector;

FIG. 2 is a schematic diagram of an example apparatus that includes an optical element receiving a pulse of laser light;

FIG. 3A is a graph showing the dose per minute produced by the example apparatus of FIG. 2, but with a laser configured to produce pulses having an energy of up to 3 mJ;

FIG. 3B is a graph showing the angular dose distribution of the example apparatus of FIG. 3A;

FIG. 3C is a graph of the dose rate produced by the example apparatus of FIG. 3A, but at different distances and pulse energies;

FIG. 3D is a graph showing the relative dose of X-ray radiation produced by the example apparatus of FIG. 3A, but filtered using aluminum plates of varying thickness;

FIG. 3E is a graph showing the attenuation coefficient of the X-ray radiation of FIG. 3D, but as a function of X-ray photon energy;

FIG. 4A is a contour graph of a simulation showing an example interaction of a pulse of laser light with a cloud of charged particles;

FIG. 4B is a graph showing an energy conversion efficiency from laser energy to electrons for the example interaction of FIG. 4A;

FIG. 4C is a contour graph showing a conical electron-beam distribution for the example interaction of FIG. 4A;

FIG. 4D is a graph showing forward and backward traveling electrons for two example laser intensities;

FIG. 5 is a schematic diagram showing five variations of the optical element of FIG. 2;

FIGS. 6A-6D are schematic diagrams showing respective variations of the example apparatus of FIG. 2, each variation having a gas cell that contains a gas medium and an optical element;

FIG. 7 is a schematic diagram of an example apparatus capable of generating ionizing radiation that includes electron, photon, and neutron radiation;

FIG. 8 is an example system that includes a gantry incorporating the example apparatus of FIG. 2;

FIG. 9A is a schematic diagram of an example optical configuration for the gantry of FIG. 8;

FIG. 9B is a schematic diagram of the example optical configuration of FIG. 9A, but in which a gantry optical element includes an aperture for passing ionizing radiation therethrough; and

FIG. 9C is a schematic diagram of the example optical configuration of FIG. 9B, but in which an optical element includes an aperture for monitoring

FIG. 9D is a schematic diagram of an example optical configuration for the gantry of FIG. 8, but in which the optical element is a high numerical aperture, off axis parabolic mirror; and

FIG. 9E is a schematic diagram of a third example optical configuration for the gantry of FIG. 8.

### DETAILED DESCRIPTION

In a general aspect, an apparatus is disclosed for generating ionizing radiation. The ionizing radiation may include electron radiation and possibly photon electromagnetic radiation (e.g., X-rays, γ-rays, etc.). In some variations, the ionizing radiation further includes neutron radiation. The apparatus may include a laser operable to produce laser light and an optical element configured to focus the laser light into a point or focus. The optical element may be coupled to a mount configured to control a position (e.g., an orientation) of the optical element relative to an optical axis. The point (or focus) may correspond to the center of an interaction region between the laser light and a gas medium (e.g., air, argon, SF₆, etc.). In some variations, the optical element is configured to focus the laser light into the point at or near the diffraction limit. In some variations, the laser and the optical element are configured such that the laser light, when focused into the point, exerts a relativistic ponderomotive force upon the gas in the interaction region. However, other physical mechanisms are possible (e.g., wakefield acceleration).

In some variations, the laser is configured to produce a pulse of laser light defined by a short pulse duration (e.g., no greater than 100 picoseconds). In some variations, the optical element is a reflective element (e.g., a parabolic mirror) having a numerical aperture no less than 0.25. In some instances, the numerical aperture is no less than 0.5 and corresponds to a high numerical aperture. During operation, the optical element may focus the laser light into the point, causing ionizing radiation to be emitted from the interaction region. In some variations, the ionizing radiation is emitted, in whole or in part, from the interaction region along a path, and as such, may form a directional ionizing radiation. The path may, for example, correspond to a single path extending outward from the interaction region. However, in some instances, a plurality of paths may extend outward from the interaction region, such as a pair of paths along opposite directions.

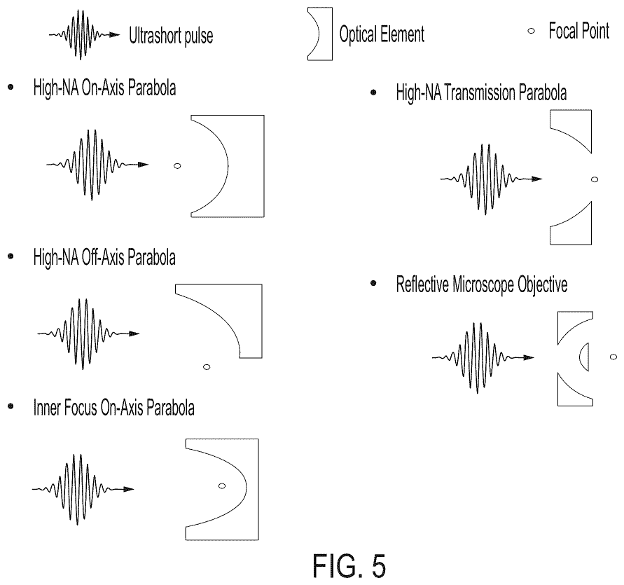

In some implementations, the pulse of laser light has a wavelength greater than 400 nm. In some implementations, the pulse of laser light has a wavelength greater than 800 nm. In some implementations, the pulse of laser light has a wavelength greater than 1 μm. In some implementations, the pulse of laser light is less than 100 femtoseconds in duration. For example, the pulse of laser light may be less than 50 femtoseconds in duration. The pulse of laser light may also be less than 20 femtoseconds. In some implementations, the numerical aperture of the optical element is at least 0.25. For example, the numerical aperture may be at least 0.5 and correspond to a high numerical aperture. In some variations, the high numerical aperture is at least 0.75. In some variations, the high numerical aperture is at least 1. The inner focus, on-axis parabolic mirror shown in FIG. 5 is one example of an optical element having a numerical aperture of at least 1. In some variations, the high numerical aperture lies in a range from 0.7 to 1.0. In some variations, the high numerical aperture lies in a range from 1.1 to 1.5.

The numerical aperture of the optical element may improve one or more operating characteristics of the apparatus as well as increase its ability to generate ionizing radiation, especially when high in magnitude. For example, the numerical aperture, when high in magnitude, may allow lower corrections for high-order Kerr effects (HOKE). The numerical aperture may also allow a B-integral to be lower in the gas medium (e.g., air), when high in magnitude. The B-integral may be a quantitative value that characterizes the distortion of a pulse wavefront, such as by non-linear effects (e.g., a non-linear index of refraction) during propagation along a path. When the B-integral is too high (e.g., greater than 2π radians), the beam may not focus optimally, which in turn, can reduce the intensity of the laser light at the focal point of the optical element. The value of the B-integral may be determined using the following equation:

$${\sum }_{i = 1}^{j} {B}_{2 i} = {\sum }_{i = 1}^{j} \frac{2 π}{λ} \int {{n}_{2 i} [ I ( z ) ]}^{i} dz \qquad ( 1 )$$

Here, λ is the pulse wavelength; n₂ᵢ are the high-order non-linear index of refraction terms (up to (2i)^{th} order) of the gas medium the pulse is propagating in; z is the propagation length; and I(z) is the pulse intensity (or laser intensity) while propagating. In configurations where the numerical aperture is high (e.g., at least 0.5), the length of the path traveled by the pulse—e.g., a length from a reflective surface of the optical element to its focal point—may be shorter due to the geometry compared to configurations where the numerical aperture is low. For the same input laser beam parameters, the B-integral can be lower if the optical element has a higher numerical aperture. The B-integral can also be lower if the wavelength of the laser light is higher. Equation (1) can also be used to determine a cumulative Kerr phase shift in the gas medium, as shown below.

$${Δϕ}_{Kerr} = {\sum }_{i = 1}^{j} {B}_{2 i} \qquad ( 2 )$$

The magnitude of Δϕ_{Kerr} may quantify distortions in a pulse wavefront that can occur as the pulse propagates in the gas medium (e.g., due to the non-linear Kerr effect).

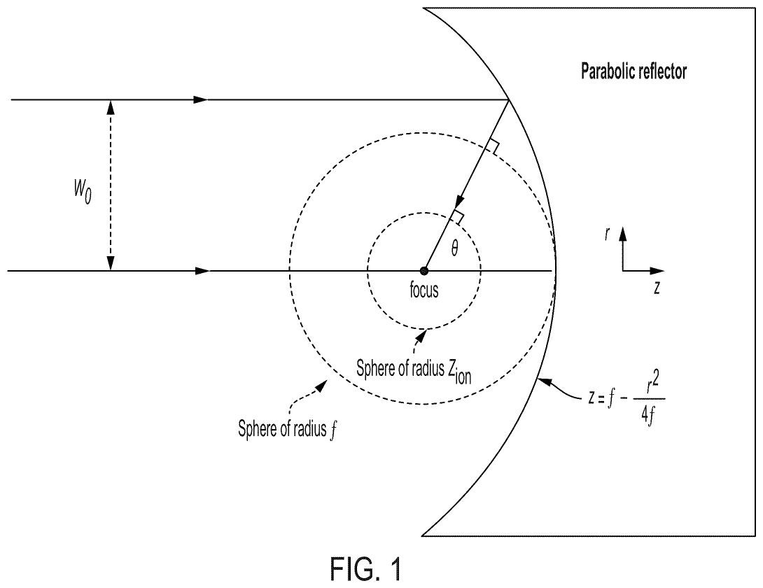

A high numerical aperture can reduce the path length from the reflective surface of the optical element up to an intensity threshold (e.g., 10¹⁴ W/cm²) at which ions are created in the gas medium. These ions may be part of a plasma in the gas medium. FIG. 1 presents a schematic diagram, in cross section, of an example optical element that includes a high numerical parabolic reflector. The high numerical parabolic reflector may have an outer sphere of radius, f, which represents an outer boundary, and an inner sphere of radius, Zᵢₒₙ, which represents an inner boundary. In some instances, the outer boundary may correspond to an extent to which the laser light interacts with the gas medium (e.g., an extent of the interaction region) and the inner boundary may correspond to a region where ion generation begins in the gas medium. Such ion generation may result in a plasma being produced in the inner sphere, and in certain cases, the plasma may extend past the inner boundary of the inner sphere (e.g., growth of the plasma by successively focusing pulses of laser light onto the interaction region).

A gas medium with an ion concentration (e.g., greater than 1%) for different ion states (e.g., +1, +2 . . . +N) can have a reduced HOKE B-integral because, for example, the non-linear index of refraction can decrease compared to a gas medium with no ion concentration. Impurities in air (e.g., atoms, molecules, etc.), which are ionized at lower intensities of laser light, can also contribute to the generation of ions in other neighboring gas molecules/atoms. Such contribution may occur in a collective manner that increases the ion concentration and thus may also assist in reducing the overall HOKE B-Integral.

In cases where the medium is isotropic (e.g., argon gas), a highly uniform intensity distribution may be formed along a surface of the outer sphere. This intensity distribution may be associated with a wavefront of the laser light and its uniformity can reduce distortions of the wavefront from non-linear effect spatially throughout the beam. For uniform intensity distributions, the B-Integral value can be the same for all of the beam surface during focusing, which will induce a constant offset for the wavefront. Beam shaping (e.g., via a deformable mirror), an apodizing filter, or both can also create a uniform intensity distribution on the surface of the outer sphere, which in turn, will reduce the overall beam wavefront distortion.

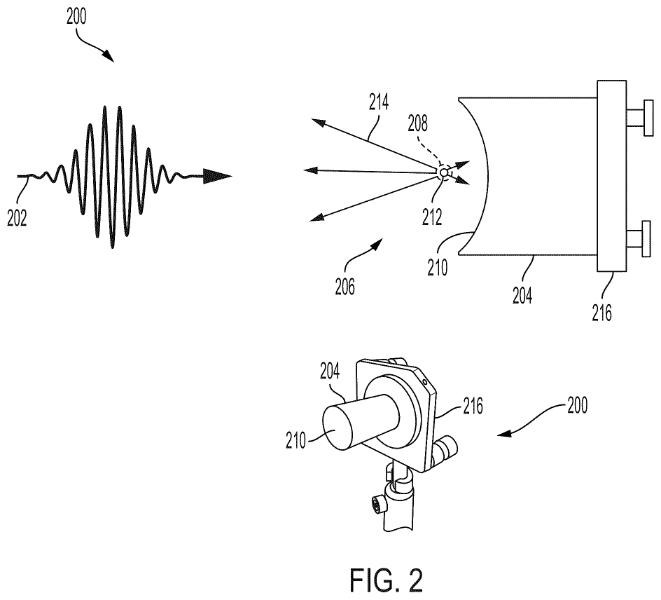

FIG. 2 presents a schematic diagram of an example apparatus 200 that includes an optical element 204 receiving a pulse of laser light 202. The optical element 204 may be, for example, a high numerical parabolic reflector, such as shown in FIG. 1. The optical element 204 includes a parabolic mirror with an aluminum reflecting surface and a numerical aperture of about 1. The parabolic mirror may, in certain cases, be formed of aluminum or an aluminum alloy. However, other types of optical elements are possible, such as shown in FIG. 5. The example apparatus 200 includes a laser (not shown) that is configured to generate the pulse of laser light 202. For example, the laser may be configured to produce the pulse of laser light 202 at a wavelength of 1.8 m and a pulse duration of 15 femtoseconds. The pulse of laser light 202 may also have other properties, such as a pulse energy of 150 μJ, a TEM00 mode, and repetition rate of 100 Hz.

The apparatus also includes a gas medium 206 having an interaction region 208 therein. The interaction region 208 may, in certain cases, be analogous to the outer sphere described in relation to FIG. 1. The gas medium 206 may be part of an ambient environment of the apparatus 200 (e.g., ambient air). However, in some variations, the gas medium 206 may be contained within a housing, such as a gas cell. The gas medium 206 may be based on a single gas component or a mixture of two or more gas components. Examples of gas components include N₂, O₂, CO₂, H₂O, He, Ar, Xe, Br₂, SF₆, WF₆, and so forth. The components, their partial pressures in the gas medium 206, and the total pressure of the gas medium 206 can be selected such that the pulse of laser light 202, when absorbed by the interaction region 208, generates a plasma whose density is below a critical plasma density (e.g., an underdense plasma). This density allows the plasma to be transmissive to a wavelength of the laser light, thus allowing the pulse of laser light to achieve a high intensity without significant plasma defocusing effects.

The optical element 204 includes a reflective surface 210 that defines a focal point 212 in the interaction region 208. The reflective surface 210 is configured to receive the pulse of laser light 202 and focus the pulse of laser light 202 at the focal point 212. In many variations, the pulse of laser light 202 is configured to generate a plasma in the interaction region 208 when focused at the focal point 212 by the reflective surface 210. In these variations, the gas medium 206 is configured emit an ionizing radiation 214 from the interaction region 208 in response to the plasma being generated therein. The ionizing radiation 214 may include electron radiation, and in certain cases, may also include photon radiation (e.g., X-rays, γ-rays, etc.). To assist the pulse of laser light 202 in generating the ionizing radiation 214, the reflective surface 210 may be configured to focus the pulse of laser light 202 at the focal point 212 at or near the diffraction limit. The reflective surface 210 may also be configured to focus the pulse of laser light 202 at the focal point 212 with a B-integral no greater than 2π radians.

In some implementations, the focal point 212 serves as a point of origin for a diffraction-limited focal spot. In these implementations, the reflective surface 210 focuses the pulse of laser light 202 at the focal point 212 at or near the diffraction limit. To do so, the reflective surface 210 may have a geometrical shape that takes into consideration characteristics of the pulse of laser light 202 (e.g., its intensity) and characteristics of the gas medium 206 (e.g., its refractive index). The reflective surface 210 may also be formed of a material that is compatible with these characteristics (e.g., aluminum), especially in regard to the high peak intensities possible for the pulse of laser light 202.

In some instances, when the pulse of laser light 202 is focused at the focal point 212 at the diffraction limit, the corresponding focal spot may have a diffraction-limited area (A) of A=λ²f²/A_{Beam}. Here, λ is the wavelength of the pulse of laser light 202, f is the focal length of the reflective surface 210, and A_{Beam} is the area of the laser beam (or pulse 202 therein). Diffraction-limited focal spots, however, may be difficult to achieve when focusing ultrashort pulses of laser light. For example, when produced in ambient air using a millijoule-class laser, the pulse of laser light 202 may experience distortions of its wavefront due to a strong non-linear Kerr effect. Plasma generation in the gas medium 206 may also destroy the integrity of the focused laser beam. In these cases, the B-integral can be minimized in order to produce a focal spot at or near the diffraction limit, which also allows for a higher peak intensity.

As shown by Equation (1), the wavelength of the pulse of laser light 202 and the non-linear refractive index of the gas medium 206 can help to determine the amount of phase shift in the pulse of laser light 202. In a gas medium, the non-linear refractive index (n₂ᵢ) typically decreases with increasing wavelength, and can dramatically decrease for higher ionization states. This latter effect can further limit the B-integral during focusing after the first ionization level. An Ammosov-Delone-Krainov (ADK) model, which models field tunnel ionization, can be used to model the ionization of molecules in air when interacting with electromagnetic radiation (e.g., the pulse of laser light 202). The first ionization of air molecules—e.g., N₂ and O₂, which are the main air constituents—is estimated by the ADK model to occur in the range of (1-2)×10¹⁴ W/cm². The corresponding cumulative Kerr phase shift is calculated to be |Δϕ_{Kerr}|<0.897 rad (less than λ₀/7) using four terms up to n₈, hence yielding minor aberrations in the pulse wavefront. This phase shift is due to the use of a relatively long central wavelength (λ₀=1.8 μm) that reduces the B-integral through the 1/λ₀ dependence, and the tight-focusing geometry that distributes the incident laser energy over a greater focusing solid angle. The ADK model also estimates the maximum ionization state for nitrogen and oxygen to be 5+ and 6+, respectively, at the calculated peak intensity of 1×10¹⁹ W/cm². At this intensity, the diffraction-limited spot size is calculated to have a diameter of ω_{FWHM}=1.0 μm. Moreover, such ionization leads to a calculated electron density of nₑ=2.65×10²⁰ cm⁻³ within a plasma of the gas medium 206, which is 23% below the critical density (nₑ=0.77n_{c}) of n_{c}=ε₀mₑω₀²/e²=3.44×10²⁰ cm⁻³ at λ₀=1.8 μm. The plasma is underdense and therefore transmissive to the laser light, allowing a high laser intensity to be achieved without significant plasma defocusing effects.

In operation, the example apparatus 200 may generate the pulse of laser light 202 by operation of the laser. The example apparatus 200 may also receive the pulse of laser light 202 at the reflective surface 210 of the optical element 204. The example apparatus 200 may additionally focus, by operation of the reflective surface 210, the pulse of laser light 202 at the focal point 212 to generate a plasma in the interaction region 208. The example apparatus 200 then emits the ionizing radiation 214 from the interaction region 208 in response to the plasma being generated therein. The ionizing radiation 214 includes electron radiation, and some variations, may include other types of ionizing radiation (e.g., photon radiation, neutron radiation, etc.).

In some implementations, the ionizing radiation 214 includes a beam of ionizing radiation. For example, the interaction region 208 may be associated with two sides, i.e., a first side facing the reflective surface 210 and a second side facing away from the reflective surface 210. The pulse of laser light 202 may enter the interaction region 208 from the first side, and the ionizing radiation 214 may exit the interaction region 208 from the second side. In this case, the ionizing radiation 214 may exit the interaction region 208 as a beam that propagates along a direction away from the reflective surface 210. In some implementations, the ionizing radiation 214 exits the interaction region 208 as two beams traveling in opposite directions. For example, and as shown in FIG. 2, the ionizing radiation 214 may exit the interaction region 208 as a first beam from the first side and as a second beam from the second side. The second beam may have an intensity significantly higher than the first beam.

In some implementations, the pulse of laser light 202 is configured to generate a relativistic ponderomotive force in the interaction region 208 when focused onto the focal point 212. The relativistic ponderomotive force may induce electrons in the plasma to move preferentially along a direction, thereby forming a beam of electron radiation. For example, the pulse of laser light may have a wavelength of 1.8 m and the gas medium 206 may be air. In this case, ionization events in the interaction region 208 may occur substantially in response to the leading edge of the pulse of laser light 202, where the field intensities can exceed 10¹⁴ W/cm². The free electrons produced by the ionization process are then driven by the relativistic ponderomotive force, where the peak pulse intensity can exceed I₀>4×10¹⁷ W/cm². This pulse intensity corresponds to the relativistic intensity threshold (near or approaching a₀=1) calculated for a pulse of laser light at 1.8 μm wavelength. For intensities above 4×10¹⁷ W/cm², electrons oscillate along the E-field polarization at velocities near the speed of light, and combined with the significant B-field that is characteristic of the relativistic regime, feel a strong v×B term from the Lorentz force. This v×B force accelerates the electrons along a direction parallel to the propagation direction of the pulse of laser light 202 at the focal point 208 (e.g., the forward direction). The accelerated electrons can then exit the interaction region 212 as a beam of ionized electron radiation.

In some implementations, the example apparatus 200 may undergo an alignment operation to ensure that the pulse of laser light 202 tightly focused at the focal point 212. In these implementations, the example apparatus may include a mount 216, such as one that allows a position of the optical element 204 (e.g., its orientation) to be selectively adjusted in rad increments. For example, the mount 216 may allow selective adjustments to one or both of a tip or tilt of the optical element 204 when coupled to the mount 216. During the alignment operation, such as shown in FIG. 2, the optical element 204 may reside in air (e.g., air at atmospheric pressure). However, other gas mediums are possible (e.g., argon, SF₆, etc.). The alignment operation may include aligning the optical element 204 along an optical axis by optimizing a signal from a radiation detector (e.g., a Geiger counter). In this case, the signal may be produced in response to the radiation detector receiving ionizing radiation 214 from the interaction region 208. In certain cases, the position of the optical element 204 is optimized when the signal from the radiation detector has a maximum magnitude. However, other criteria are possible.

In some implementations, the example apparatus 200 may be aligned using operations that include determining a target position of the reflective surface 210 that corresponds to a maximum intensity of the ionizing radiation 214. The target position may be based on data that includes measured intensities of the ionizing radiation 214 and respective positions of the reflective surface 210. After the target position is determined, the optical element 204 may be secured (e.g., via the mount 216) to set the reflective surface 210 in the target position. In some implementations, the operations also include propagating the pulse of laser light 202 along an optical axis that terminates at the reflective surface 210. In these implementations, determining the target position of the reflective surface 210 includes displacing the reflective surface 210 to alter a position of its focal point 212 relative to the optical axis. Determining the target position also includes measuring an intensity of the ionizing radiation 214 emitted from the interaction region 208 when the focal point 212 is in the altered position.

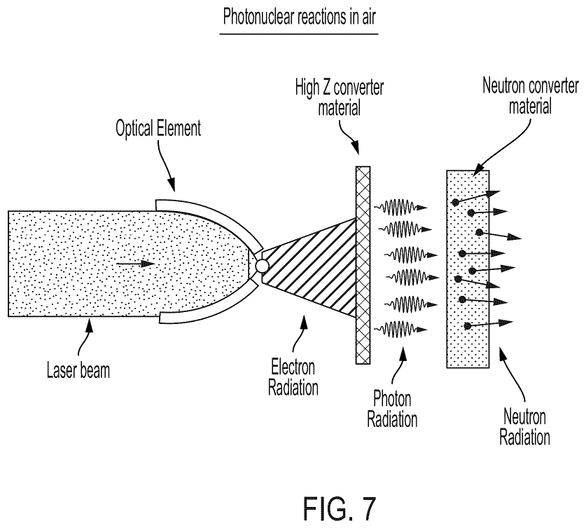

In many variations, the ionizing radiation 214 includes electron radiation. However, as noted above, the ionizing radiation 214 may also include photon radiation, such as X-rays and γ-rays. For example, photon radiation may be emitted from the gas medium 206 (e.g., air, argon, SF₆, etc.) in which the interaction region 208 resides. The photon radiation may also be emitted from an electron-to-photon converter (not shown) of the example apparatus 200. For example, the example apparatus 200 may include an electron-to-photon converter adjacent the interaction region 208. The electron-to-photon converter may be formed of a material having a high atomic number (Z), such as copper or tungsten. In further variations, the ionizing radiation 214 includes neutron radiation. In these variations, the example apparatus 200 may include a photon-to-neutron converter, and the electron-to-photon converter may reside between the interaction region 208 and the photon-to-neutron converter. Examples of the electron-to-photon converter and the photon-to-neutron converter are described further in relation to FIG. 7.

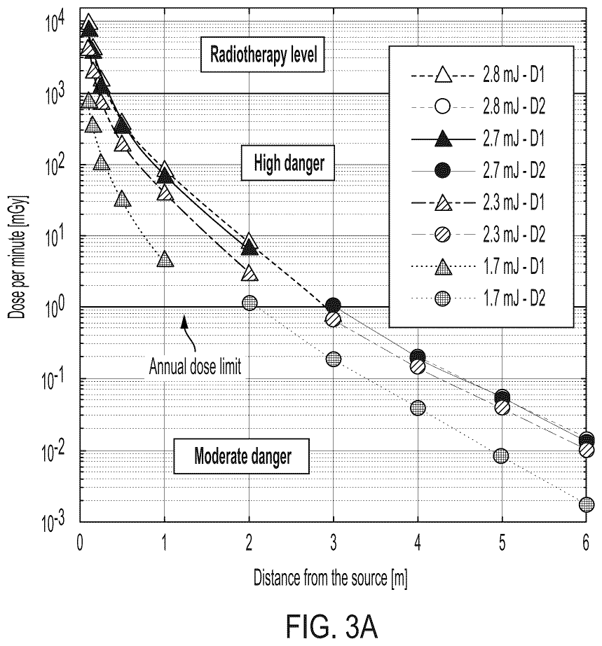

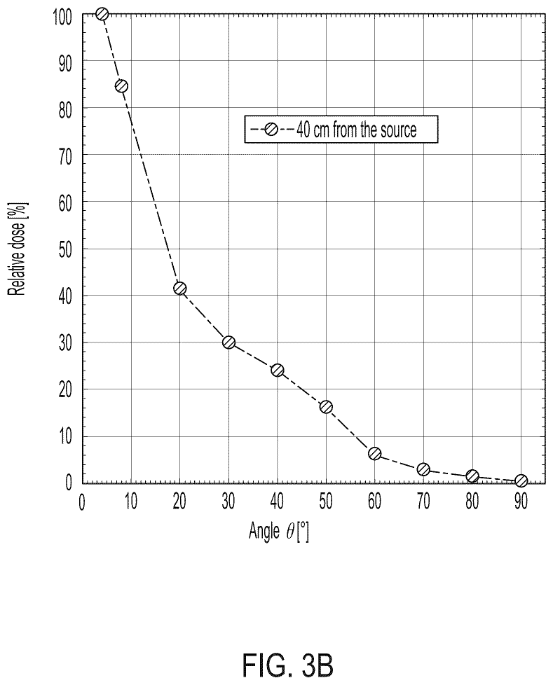

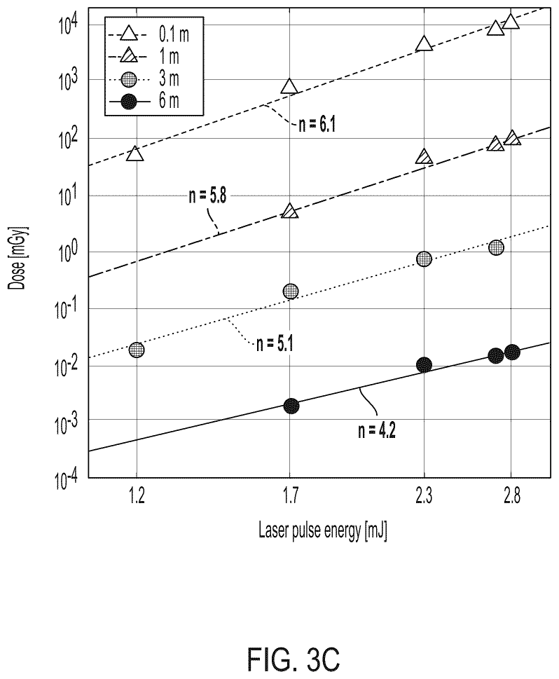

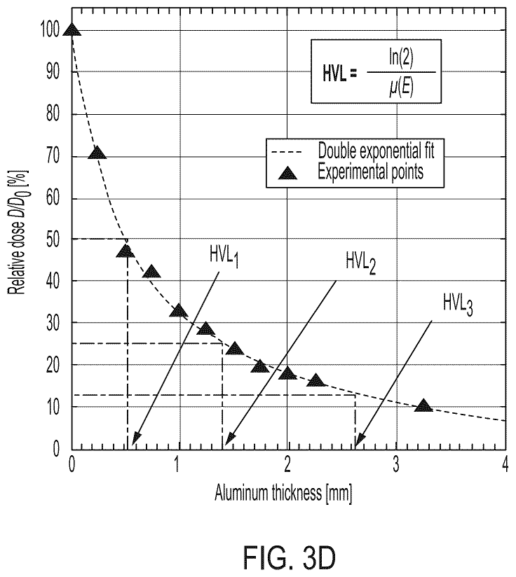

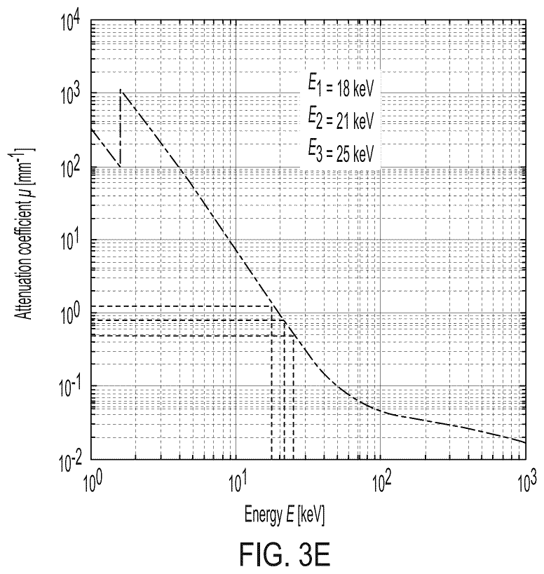

In some implementations, an intensity of the ionizing radiation 214 emitted from the interaction region 208 may be increased by increasing an intensity of the pulse of laser light 202. For example, the laser of the example apparatus 200 may produce a pulse of laser light having a pulse energy of up to 3 mJ. FIG. 3A presents a graph showing the dose per minute produced by the example apparatus 200 of FIG. 2, but with a laser configured to produce pulses of up to 3 mJ in energy. At distances up to 25 cm from the interaction region 208, FIG. 3A shows the dose rate under optimal conditions to be above radiotherapy levels. This dose rate corresponds to air at atmospheric pressure (e.g., the optical element 204 resides in air at atmospheric pressure). The ionizing radiation 214 emitted from the interaction region 208 is directional, as shown by FIG. 3B. The ionizing radiation 208 includes high energy electrons along with a secondary contribution of X-ray radiation. The secondary contribution of X-ray radiation may be produced by the high energy electrons via a Bremsstrahlung mechanism. The dose rate indicates a conversion efficiency from laser light (or photons thereof) to ionizing radiation in the range from 0.001%-0.01%. FIG. 3C presents a graph of the dose rate produced by the example apparatus of FIG. 3A, but at different distances and pulse energies.

The interaction between the pulse of laser light 202 and the air may include multiple mechanisms, such as a pondermotive force mechanism and a laser wakefield acceleration mechanism induced by the electromagnetic field of the laser light. However, use of the laser wakefield acceleration mechanism typically requires long focal lengths (e.g., numerical apertures of 0.1 or lower), and as such, the pulse of laser light 202 may become highly distorted. This distortion can result from non-linear effects during the propagation of the pulse of laser light 202 to the interaction region 208. The laser wakefield acceleration mechanism may, in certain cases, be smaller in contribution than the pondermotive force mechanism.

In some implementations, the example apparatus 200 tightly focuses pulses of laser light 202 that have long wavelengths (e.g., greater than 1 μm). One or both of a tight focusing and a long wavelength may prevent the pulses of laser light 202 from accumulating significant non-linear distortion effects and high order Kerr effect (HOKE) distortions, such as by keeping the pulses of laser light 202 below a threshold propagation length. The pulses of laser light 202 may thus be able to reach high intensity at the interaction region during focusing. The high intensity of the focused pulses may increase an intensity of the ionizing radiation 214 emitted from the interaction region 208. However, other contributing factors are possible. Examples include the laser central wavelength, the laser beam mode, the laser pulse energy, the wavefront shape, the pulse repetition rate, the laser pulse duration, the laser contrast, the gas pressure, and the gas type. In some variations, the laser is configured to produce laser light at infrared wavelengths. Infrared wavelengths may allow the wavefronts associated with each short pulse to be less susceptible to imperfections in the optical element.

In some implementations, the example apparatus 200 includes an optical pathway that extends between the laser and the optical element 204. In these implementations, the example apparatus 200 also includes a second optical element disposed on the optical axis. The second optical element includes a deformable reflective surface and a transducer coupled to the deformable reflective surface. The transducer is configured to selectively deform the deformable reflective surface in response to receiving a signal that represents a target shape of the deformable reflective surface. Such deformation may alter a wavefront of the pulse of laser light 202 as it propagates along the optical path. This alteration may counteract distortions of the pulse of laser light 202 (e.g., HOKE distortions) that can occur to the pulse of laser light 202 before reaching the focal point 212. In certain cases, the deformation of the deformable reflective surface may allow the second optical element to shape the wavefront of pulse of laser light 202 to a target profile. As such, when the pulse of laser light 202 reaches the focal point 212, its wavefront may have a profile that maximizes an intensity of ionizing radiation 214 that is emitted from the interaction region 208.

In many implementations, the example apparatus 200 generates electron radiation. In these implementations, the electron radiation may generate a secondary emission of X-ray electromagnetic radiation, such as through the Bremsstrahlung mechanism. Such X-ray radiation may be filtered using a plate of aluminum. FIG. 3D presents a graph showing the relative dose of X-ray electromagnetic radiation produced by the example apparatus of FIG. 3A, but filtered using aluminum plates of varying thickness. The aluminum plates may attenuate the X-ray photons reaching an X-ray detector. The attenuated intensity, in percent, is shown on the ordinate of the graph, with 0% corresponding to the absence of an aluminum plate. FIG. 3E presents a graph showing the attenuation coefficient of the X-ray electromagnetic radiation of FIG. 3D, but as a function of X-ray photon energy.

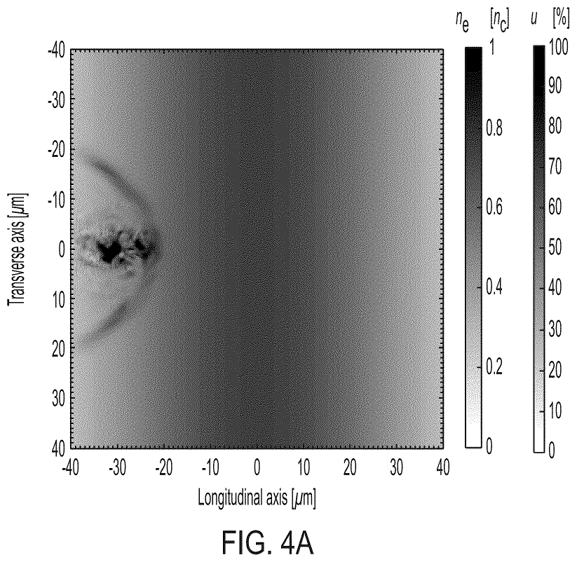

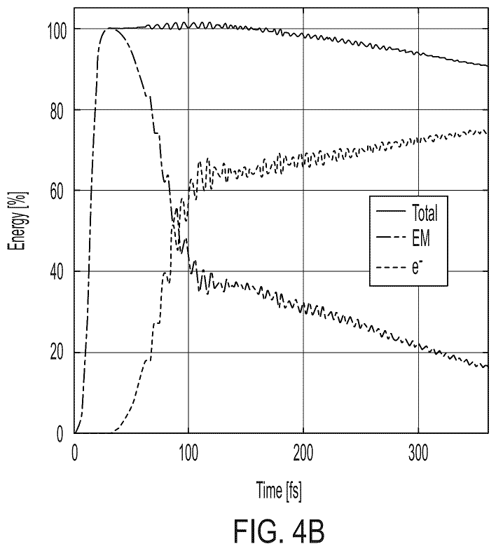

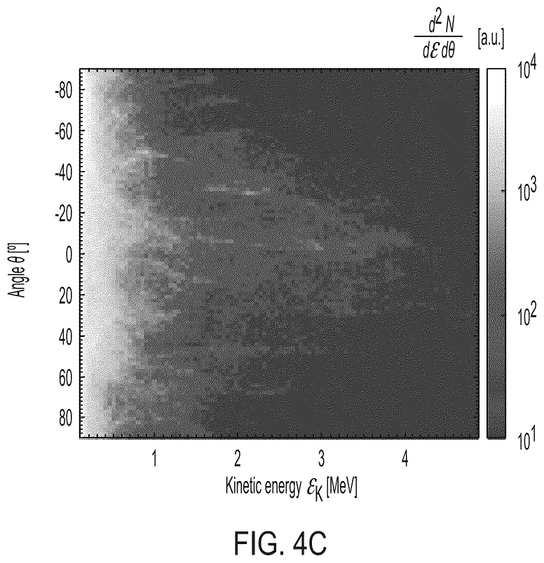

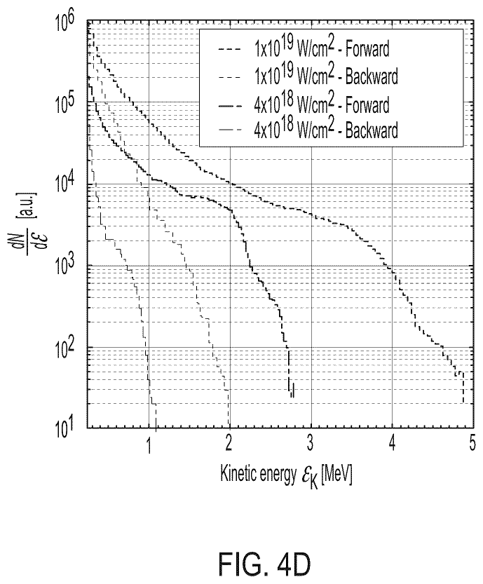

Now referring to FIG. 4A, a contour graph is presented of a simulation showing an example interaction of a pulse laser light with a cloud of charged particles. The simulation corresponds to a particle-in-cell (PIC) simulation and may represent the physical processes that can occur in the interaction region. PIC simulations can be used for modeling high-intensity laser interactions with a cloud of charged particles, commonly referred to as a plasma. To perform this task, the underlying program code solves the relativistic Newton equations of movement, coupled with the Maxwell equations, to model the propagation of electromagnetic (EM) fields through the plasma. This process can be discretized over either a 1D, 2D, or 3D numerical mesh grid, and the equations are solved for each time step required.

To simulate the example interaction, an electromagnetic wave (e.g., a pulse of laser light) is inserted in the 2D simulation box from the left side of the graph, as defined using parameters of the laser. The initial plasma distribution is selected to represent the experiment as realistically as possible. Execution of the program code serves to propagate the electromagnetic wave (or waves) through the plasma and takes into account ionization events. Such events may include collisions between charged particles, electromagnetic fields generated from moving charged particles, and other kinds of particle interactions, such as Bremsstrahlung and pair production.

The contour graph of FIG. 4A shows the propagation of the electromagnetic wave into the plasma, forming a large electromagnetic shockwave in dark grey. This conical wave arises from high energy electrons driven by the pulse of laser light. FIG. 4B presents a graph showing an energy conversion efficiency from laser energy to electrons for the example interaction of FIG. 4A. This conversion efficiency is high, which may be due to the near-critical plasma density that enhances the laser-matter interactions. FIG. 4C presents a contour graph showing a conical electron-beam distribution for the example interaction of FIG. 4A. The contour graph shows a dependence of this distribution on kinetic energy. As shown by FIG. 4C, the highest energy electrons form a narrower cone than the low energy electrons. Finally, FIG. 4D presents a graph showing forward and backward traveling electrons for two example laser intensities. The electron spectrum follows a Maxwell-Boltzman distribution. Moreover, the ratio of maximum energies in the two simulations matches the ratio of the measured and theoretically estimated maximum energies from the relativistic ponderomotive force.

Now referring back to FIG. 2, the example apparatus 200 may be used in a system, such as a system for radiotherapy, food and equipment sterilization, X-ray imaging, radiation stress testing of devices, and so forth. In these applications, the laser of the example apparatus 200 may be configured to produce a pulse repetition rate of 100 KHz. In this configuration, the laser may allow the example apparatus 200 to produce dose per minute in the range of kiloGrays (kGy). A system delivering this amount of dose would be of high benefit in radiotherapy, food and equipment sterilization, X-ray imaging and radiation stress testing of devices. Furthermore, the sub-nanosecond temporal duration of the emitted ionizing radiation could possibly produce a “flash dose effect” on biological targets during the irradiation process. Due to the example apparatus 200 having a simple configuration and operation, the system may be more straightforward to implement than other laser-based X-ray sources.

The example apparatus 200 brings multiple advantages relative to traditional devices for generating ionizing radiation. For example, the example apparatus 200 does not require the use of a vacuum chamber. The example apparatus 200 also does not require an in-vacuum gas jet, liquid jet, or solid target at the interaction region 208 to generate ionizing radiation. The example apparatus 200 additionally has a simple and compact design. In some variations, the example apparatus 200 can convert laser energy to ionizing radiation with a notable efficiency (e.g., 0.001% to 0.01%). The apparatus can also allow an electron-to-photon converter to be positioned very close to the interaction region 208. Such positioning may permit the example apparatus 200 to generate ionizing radiation whose photon radiation consists only of short-pulse X-ray photons. In some variations, the example apparatus 200 produces ionizing radiation that is directional and divergent, thereby allowing uniform dose distributions for irradiation. The example apparatus 200 can also produce a short pulse (e.g., below 100 picoseconds) of ionizing radiation that provides an ultra-high, virtually instantaneous dose rate (e.g., greater than 10⁷ Gy/s). The example apparatus 200 additionally can avoid anode degradation and debris. Furthermore, the example apparatus 200 can be easily scaled to higher doses, electron energies, and photon energies. Radiation shielding is also easy due to the directionality and energy level of the ionizing radiation 214.

In some implementations, the optical element 204 of the example apparatus 200 is formed of a high-Z material (e.g., copper, tungsten, etc.) to increase X-ray production. The high-Z material may include atoms whose atomic number is higher than 25. In some instances, the atomic number may be higher than 35. In some implementations, the example apparatus 200 includes a body formed of high-Z material that is close to the interaction region 208. The body may be operable to convert electrons to photon radiation (e.g., X-rays, γ-rays, etc.) and thus serve as an electron-to-photon converter.

In some implementation, the optical element 204 is an elliptical mirror and the laser is configured to produce an input focused pulse. In some implementations, the optical element 204 is a focusing parabola. Examples of the focusing parabola include an off-axis focusing parabola, a transmission parabola, and an on-axis parabola. The focusing parabola may, in some instances, have a high numerical aperture (e.g., greater than 0.25). The focusing parabola may, in some instances, have a focus within a cavity defined by its reflective surface. In some implementations, the optical element 204 is a reflective microscope objective. FIG. 5 presents a schematic diagram showing five possible variations of the optical element 204 of FIG. 2.

In some implementations, the example apparatus 200 generates ionizing radiation in the form of an electron beam by the focusing an infrared ultrashort laser pulse with a high-numerical aperture optical element. The ionizing radiation 214 may be used in radiotherapy where the electron beam itself, or a secondary beam of X-rays produced through the Bremsstrahlung mechanism, is used to treat a medical condition. As evidenced by FIG. 3A, experimental measurements show the example apparatus 200 can be configured to reach the average dose levels used in conventional radiotherapy. Additionally, the example apparatus 200 may be useful for ‘FLASH radiotherapy’, which could advantage of the sub-nanosecond temporal duration of ionizing radiation produced in certain configurations of the example apparatus 200. In some variations, the example apparatus 200 is configured to generate an electron beam having electrons with an energy up to 1 MeV. In some variations, the example apparatus 200 is configured to generate an electron beam having electrons with an energy up to 5 MeV. In some variations, the example apparatus 200 is configured to generate an electron beam having electrons with an energy up to 10 MeV. Other energy levels are possible.

The dose to laser-pulse energy of the example apparatus 200 may have a dependence of up to the power 6 (e.g., see FIG. 3A). In some variations, the example apparatus 200 may allow the dose generation to be greater if the laser is configured to produce cycle pulses at a longer wavelength and higher energy. In some variations, the example apparatus 200 includes a gas medium 206 having more electrons per atom or molecule than air. The gas medium 206 could, for example, include an atom whose total electrons number greater than seven. The gas medium 206 could also, for example, include a molecule whose total electrons number greater than 14. In these cases, the gas medium 206 could raise the number of available electrons to be accelerated, and thus raise the amount of ionization radiation 214 generated by the example apparatus 200.

In some implementations, the example apparatus 200 is configured to compete with flash radiotherapy sources and micron size X-ray sources. In these implementations, the example apparatus 200 could tune the maximum electron energy and generated dose at each laser shot by varying the laser parameters, such as laser energy, laser pulse duration, and the beam mode. The example apparatus 200 could also have a mobile source due to the compactness of the laser energy to ionization radiation converter. Additionally, the example apparatus 200 could separate the laser from the interaction region 208 because it is typically easier to propagate a laser beam than a particle beam, an X-ray beam, or a γ-ray beam. In this case, a flash radiotherapy facility based on the example apparatus 200 (or system incorporating the example apparatus 200) could have a single central laser that emits and propagates laser light to many different therapy rooms. Furthermore, the example apparatus 200 could produce a flash (e.g., a very short X-ray and electron pulse) of sub-nanosecond duration depending on the electron energy, current, and distance.

In some implementations, the example apparatus 200 includes an electron-to-photon converter (e.g., X-ray photons, γ-ray photons, etc.). In these implementations, the use of high energy photons (e.g., X-rays) as the form of ionizing radiation can be advantageous because of their penetration depth compared to electrons. A Bremsstrahlung mechanism may be used to convert an electron beam into an X-ray source through the electron-to-photon converter. The electron-to-photon converter may be formed of high-Z materials, such as copper and tungsten. These materials can be placed in the electron beam and can be of any geometric shape and size, as the electron source for the apparatus may be on the order of the size of the focal volume (e.g., cubic microns). In many variations, the electron-to-photon converter is positioned near the interaction region 208, which may also reduce the size of the example apparatus 200. However, the electron-to-photon converter could also be positioned at a sufficient distance from the interaction region to avoid ablation by the focused laser light. Pulses in the focused laser light may, in certain cases, damage the electron-to-photon converter and thus necessitate it being placed far enough from the interaction region 208 to provide sufficient divergence of the optical beam. In some variations, a shadow can be created in the beam that allows the electron-to-photon converter to be placed in close proximity to the focal spot.

In some variations, the electron-to-photon converter is a foil. In these variations, a m-to-mm thick foil may be brought directly into the electron beam. In some variations, the electron-to-photon converter is m-to-mm sized sphere or ball. To minimize blockage of the incoming pulse of laser light—especially if the example apparatus 200 includes an on-axis parabola—a small m-to-mm sized ball can be brought close to the focal point 212 and electron beam. In some variations, the electron-to-photon converter is a tube. In these variations, the electron beam is incident inside tube, which subsequently converts the electrons in the beam of ionizing radiation to X-ray radiation. The geometry of tube may also help with collimation of the X-ray radiation emitted from the interaction region 208. In some variations, the electron-to-photon converter is a trapped particle. In such variations, a nano- or micro-sized particle can be suspended in an optical trap using the same high numerical aperture optical element. The equilibrium position of the trapped particle may be half a Rayleigh length away from the focal point 212. Thus, the trapped particle could be very close to the interaction region 208 (e.g., the source of electron radiation).

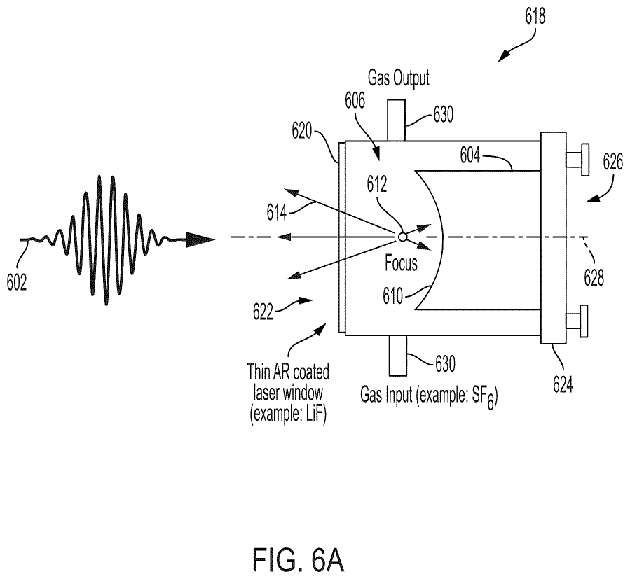

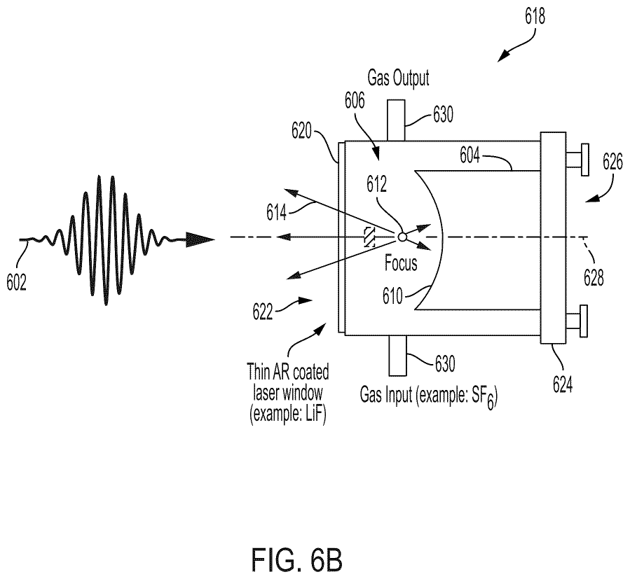

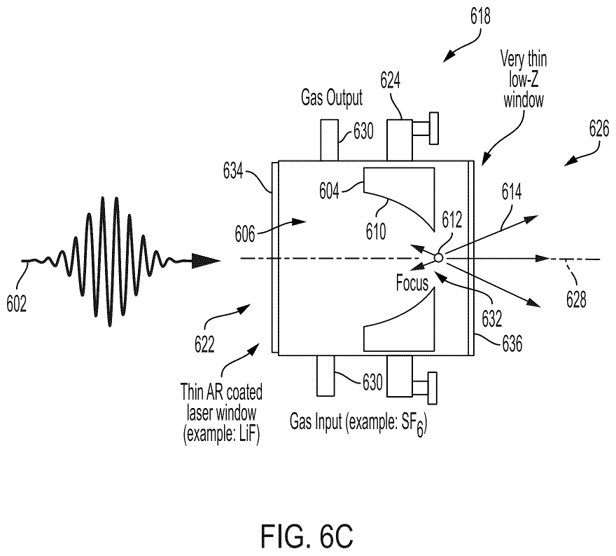

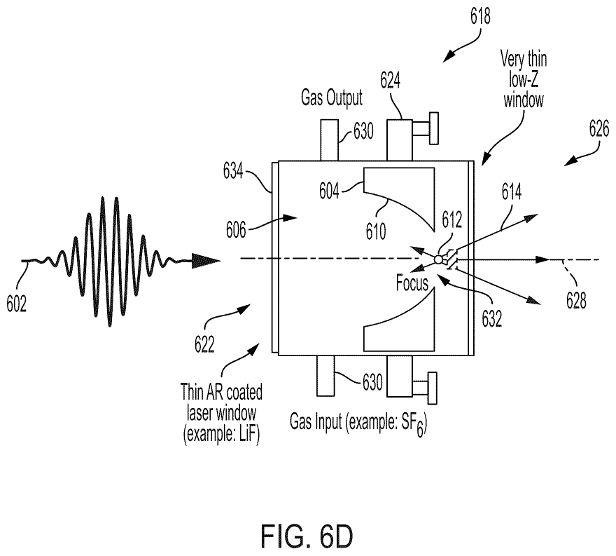

In some implementations, the gas medium 206 includes a gaseous atom that has a high number of electrons per atom (e.g., greater than 7) or a gaseous molecule that has a high number of electrons per molecule (e.g., greater than 14). Examples of such atoms and molecules include Ar, Xe, and SF₆. Mixtures of two or more gas components are possible (e.g., He and SF₆). The gas medium 206 may, in certain instances, have a non-linear index of refraction comparable to air. Moreover, the non-relativistic or relativistic plasma effects due to the free electron density in the gas medium 206 may induce minimal beam distortion or energy loss during propagation to the focal point 212. In some implementations, the example apparatus 200 resides in ambient environment of the gas medium 206. In other implementations, the example apparatus 202 includes a housing that defines an enclosed volume for the gas medium 206. The housing may, for example, correspond to a gas cell. FIGS. 6A-6D present schematic diagrams showing respective variations of the example apparatus 200 of FIG. 2, each variation having a gas cell that contains the gas medium 206 and the optical element 204. Features common to both FIG. 2 and FIGS. 6A-6D are related via coordinated numerals that differ in increment by four hundred.

In FIGS. 6A and 6B, the gas cell 618 may include a window 620 transparent to the pulse of laser light 602 and the ionizing radiation 614. For example, the window 620 may formed of LiF and have an anti-reflective coating disposed on its exterior surface. In these implementations, the window 620 is disposed on a first side 622 of the gas cell 618. The gas cell 618 then also includes a mount 624 on a second side 626 of the gas cell 618 opposite the first side 622. The mount 624 is coupled to the optical element 604 and configured to selectively alter a position of the optical element 604 (e.g., its orientation) relative to an optical axis 628 of the gas cell 618. The optical element 604 is coupled to the mount 624 such that the reflective surface 610 faces the window 620. In some variations, such as shown in FIGS. 6A and 6B, the gas cell 618 includes a port 630 for exchanging the gas medium 606 with the gas cell 618. For example, the gas cell 618 may include a pair of ports 630 for, respectively, supplying the gas medium 606 to and discharging the gas medium 606 from the gas cell 618. In FIG. 6B, the gas cell 618 includes an electron-to-photon converter that is disposed adjacent the focal point 612. The electron-to-photon converter may be formed of a high-Z material, such as copper or tungsten.

In some implementations, the gas cell 618 includes multiple windows. For example, in FIGS. 6C and 6D, the reflective surface 610 of the optical element 604 has a parabolic shape and includes an aperture 632 at a vertex of its parabolic shape. In this configuration, the optical element 604 may correspond to a transmission parabola (e.g., a transmission parabola of high numerical aperture). The gas cell 618 includes a first window 634 transparent to the pulse of laser light and disposed on the first side 622 of the gas cell 618. The first window 634 may, in certain cases, also be transparent to the ionizing radiation 614. So, for example, the first window 634 may formed of LiF and have an anti-reflective coating disposed on its exterior surface. However, other materials and coatings are possible. The gas cell 618 also includes a second window 636 transparent to the ionizing radiation 614 and disposed on the second side 626 of the gas cell 618 opposite the first side 622. The second window 636 may be formed of a low-Z material, such as beryllium, diamond, quartz, and LiF. The gas cell 618 additionally includes a mount 624 disposed between the first and second windows 634, 636 and coupled to the optical element 604. The mount 624 is configured to selectively alter a position of the optical element 604 (e.g., its orientation) relative to the optical axis 628 of the gas cell 618. In these implementations, the optical element 604 is coupled to the mount 624 such that the reflective surface 610 faces the first window 634 and the aperture 636 faces (or is closest to) the second window 636. Similar to FIGS. 6A and 6B, the gas cells of FIGS. 6C and 6D may include a port 630 for exchanging the gas medium 606 with the gas cell 618. However, in FIG. 6D, the gas cell 618 includes an electron-to-photon converter that is disposed adjacent the focal point 612. The electron-to-photon converter may be formed of a high-Z material, such as copper or tungsten.

Now referring back to FIG. 2, the gas medium 206 may, in certain variations, includes a gaseous molecule that has a high number of electrons per molecule (e.g., greater than 14). In these variations, the gaseous molecule may include an atom whose atomic number (Z) is at least 35. Examples of the gas molecule include Br₂, WF₆, and UF₆. In these variations, the gas medium 206 can provide electrons for ionization, such as by the pulse of laser light, and further serves as an electron-to-photon converter. In some variations, the gas medium 206 includes a mixture of first and second gas components. The first gas component may be a gas that has a high number of electrons per atom or molecule (e.g., 02), and the second gas component may be gas that includes an atom whose atomic number is greater than 35 (e.g., Xe).

In some implementations, the example apparatus 200 includes a photon-to-neutron converter. The photon-to-neutron converter may, for example, be a neutron converter material. FIG. 7 presents a schematic diagram of an example apparatus capable of generating ionizing radiation that includes electron, photon, and neutron radiation. In FIG. 7, the optical element of the example apparatus is configured as a transmission parabola. However, other configurations are possible. The focal point of the transmission parabola defines an interaction region in a gas medium (e.g., air) that emits electron radiation along a direction that defines an emission pathway. The electron radiation may include electrons of high energy (e.g., at least 1 MeV). For example, the electron radiation may include electrons having an energy of at least 5 MeV. In some instances, the electrons have an energy of at least 10 MeV. To generate high energy electron radiation, the laser of the example apparatus may, for example, have an energy of at least 1 mJ with an emission wavelength in an infrared spectrum (e.g., 0.7 μm-15 μm).

For instance, using 200 mJ of laser pulse energy tightly focused in air, the B-integral can be low enough to reach an intensity high enough to generate an electron ponderomotive energy of about 15 MeV, which in turn, can enable the generation of Bremsstrahlung photons with energies up to 15 MeV. These photons can then be further converted into neutrons via a photonuclear reaction. Such a conversion reaction becomes possible when the Bremsstrahlung photons reach an energy of about 10 MeV (or greater). The conversion of Bremsstrahlung photons into neutrons can allow the production of ultrashort pulses of neutrons (or beams thereof) in ambient air.

The example apparatus includes an electron-to-photon converter that is positioned on the emission pathway adjacent (e.g., downstream) the focal point of the optical element (or adjacent the interaction region). The electron-to-photon converter allows electron radiation from the interaction region to be captured and converted into photon radiation. This process may involve absorption of the electron radiation, such as by a high-Z gas, or as shown in FIG. 7, a high-Z material. The photon electromagnetic radiation may include high-energy X-ray photons (e.g., greater than 10 keV), γ-rays, or both. The photon energies may be broadband and may range up to the maximum electron energy.

The photon-to-neutron convertor is positioned on the emission pathway adjacent (e.g., downstream) the electron-to-photon converter. The photon-to-neutron convertor is operable to capture photon radiation emitted from the electron-to-photon convertor, and after capture, generate neutron radiation. The neutron radiation may propagate, in whole or in part, along the emission pathway away from the optical element and converters. The photon-to-neutron converter may include atoms having a photonuclear (γ, n) cross section. Examples of such atoms include ²H, ¹²C, ²⁷Al, ⁶³Cu, ²⁰⁸Pb, and ²³⁵U. In some instances, the photonuclear cross section has a lower limit. For example, the lower limit may be 1 millibarn for photon radiation (or energies thereof) emitted from the electron-to-photon convertor. For instance, the photonuclear giant resonance cross section of hydrogen gas as a converter is about 2.7 millibarns and can range up to 1200 millibarn for ²³⁶U.

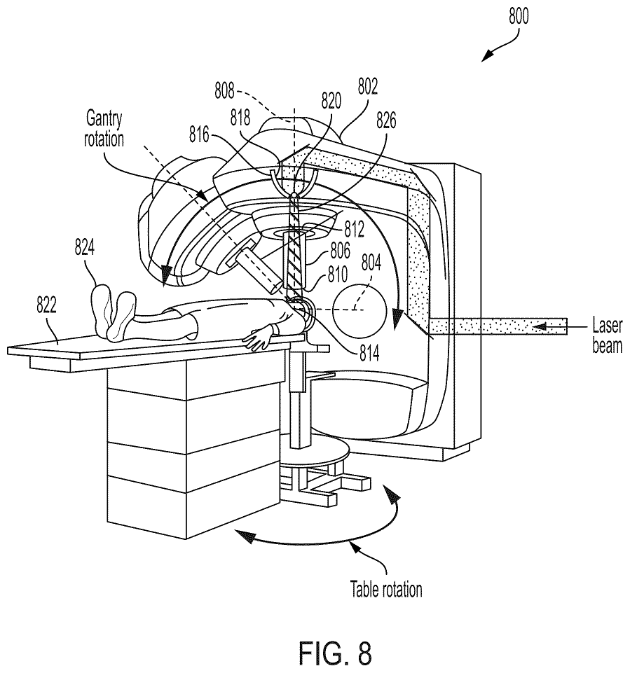

Now referring back to FIG. 2, the example apparatus 200 may, in some implementations, be part of a system, such as part of an all-optical rotating gantry for irradiating a target (e.g., person). The system may be in air. For example, FIG. 8 presents an example system 800 that includes a gantry 802 incorporating the example apparatus 200 of FIG. 2. The example system includes a gantry housing that defines a shape of the gantry and a motor to rotate the gantry 802 about a gantry axis 804. The gantry housing may enclose the example apparatus 200 and one or more gantry optical elements (e.g., mirrors, lenses, filters, etc.). The one or more gantry optical elements may define an optical path for the pulse of laser light 202 to reach the optical element 204 of the example apparatus 200. In FIG. 8, the optical path is shown with dotted hatching. In some instances, the gantry housing encloses the laser of the example apparatus 200. In these instances, the optical path may be entirely enclosed in the gantry housing. In other instances, the laser is located remotely (e.g., in a room different than the gantry housing). In such instances, a portion of the optical path may reside exterior to the gantry housing.

In some implementations, the example system 800 includes a laser configured to generate a pulse of laser light. The example system 800 also includes a gantry 802 configured to rotate about a gantry axis 804. The gantry 802 includes a collimator 806 extending along a collimator axis 808 between first and second collimator ends 810, 812. The collimator axis 808 intersects the gantry axis 804 at an isocenter 814 of the example system 800, and the first collimator end 810 is configured to face the isocenter 814 while the gantry 802 rotates about the gantry axis 804. As shown in FIG. 8, the isocenter 814 is the point in space where the collimator axis 808 intersects the gantry axis 804; as such, the location of the isocenter 814 may be defined by the geometric arrangement of the collimator 806 relative to the gantry 802. The gantry 802 also includes an optical element 816 proximate the second collimator end 812 and comprising a reflective surface that 818 defines a focal point 820 in a gas medium. The reflective surface 818 is configured to receive the pulse of laser light and focus the pulse of laser light at the focal point 820. The gas medium is adjacent the reflective surface 818 and has an interaction region therein. The interaction region includes the focal point 820. The example system 800 additionally includes a support surface 822 (e.g., an adjustable table) that is configured to support a target 824 (e.g., a person) and position at least a portion of the target 824 at the isocenter 814 of the example system 800.

During operation, the laser generates the pulse of laser light, which generates a plasma in the interaction region when focused at the focal point 820. In response, the gas medium emits a beam of ionizing radiation 826 from the interaction region. In FIG. 8, the beam of ionizing radiation 826 is shown with cross hatching. The beam of ionizing radiation 826 propagates towards the second collimator end 812. The collimator 806 may align the beam of ionizing radiation 826 (or a portion thereof) such that the beam of ionizing radiation 826 propagates along the collimator axis 808 towards the isocenter 814. The target 824 then receives the beam of ionizing radiation 826 in a portion residing at the isocenter 814. Also during operation, the gantry 802 may rotate about the gantry axis 804, thereby rotating at least a portion of the collimator axis 808 about the gantry axis. This rotation also alters an angle at which the beam of ionizing radiation 826 is incident upon the isocenter 814 and thus the target 824.

In some implementations, the support surface 822 is defined by a table. The table may be configured to selectively position a portion of the target 824 at the isocenter 814. For example, the table may be configured to rotate the target 824 about a table axis. In doing so, the table may assist the example system 800 in selectively positioning the target 824 for irradiation. In some variations, such as shown in FIG. 8, the example system 800 is a medical system, and the table is configured to support and rotate a person. However, other types of systems are possible.

In some implementations, the example apparatus 800 includes the gantry housing and a plurality of gantry optical elements internal to the gantry housing. The plurality of gantry optical elements are configured to define at least part of an optical path between the laser and the optical element 816. In some variations, the laser is disposed in the gantry housing and the optical path is internal to the gantry housing. In these variations, the plurality of gantry optical elements may define the entire optical path. In other variations, the gantry housing includes an optical port configured to receive the pulse of laser light from the laser. The optical path includes a portion internal to the gantry housing, and the portion may extend between the optical port and the optical element 816. In such variations, the plurality of gantry optical elements may define only part of the optical path.

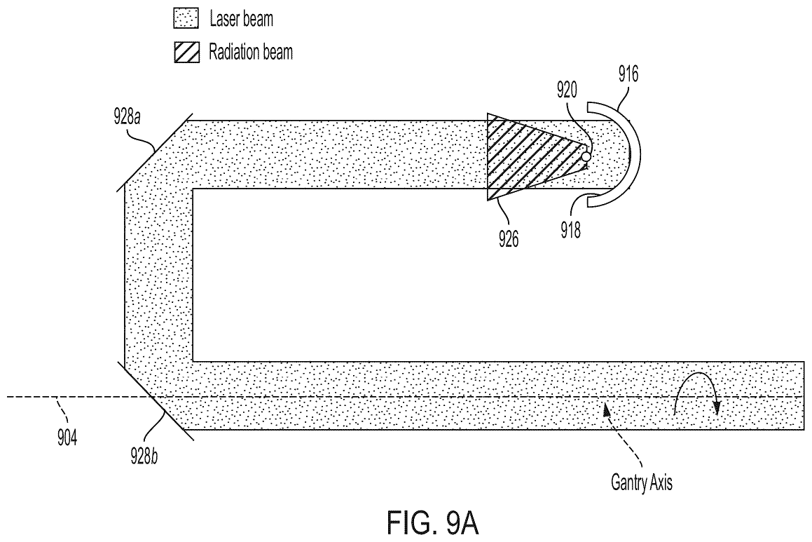

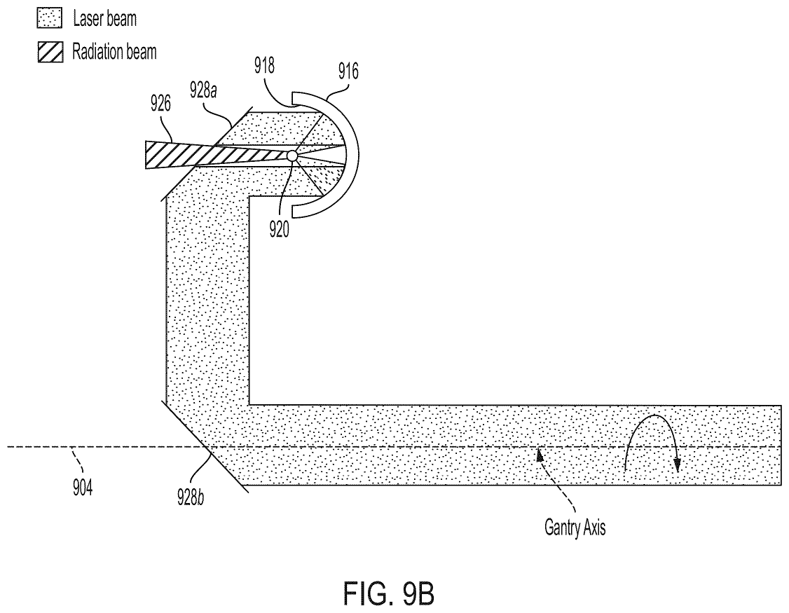

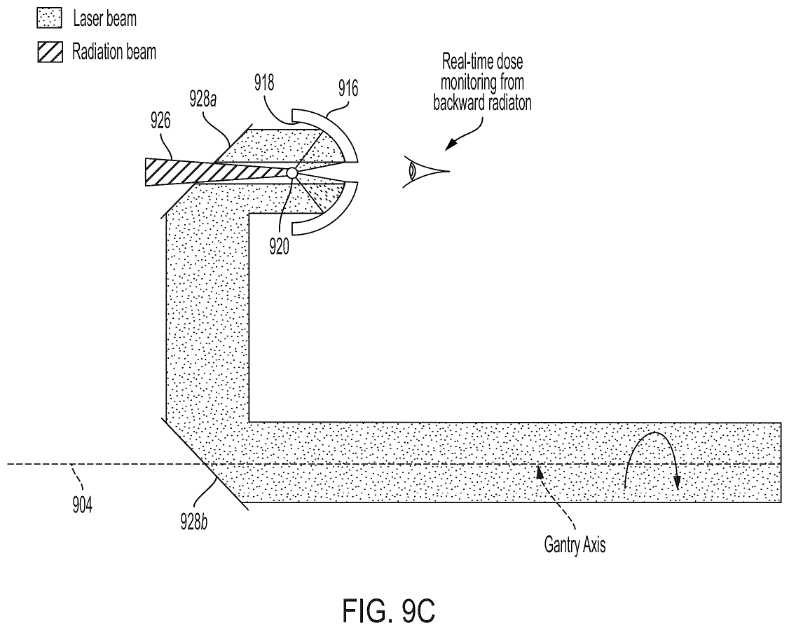

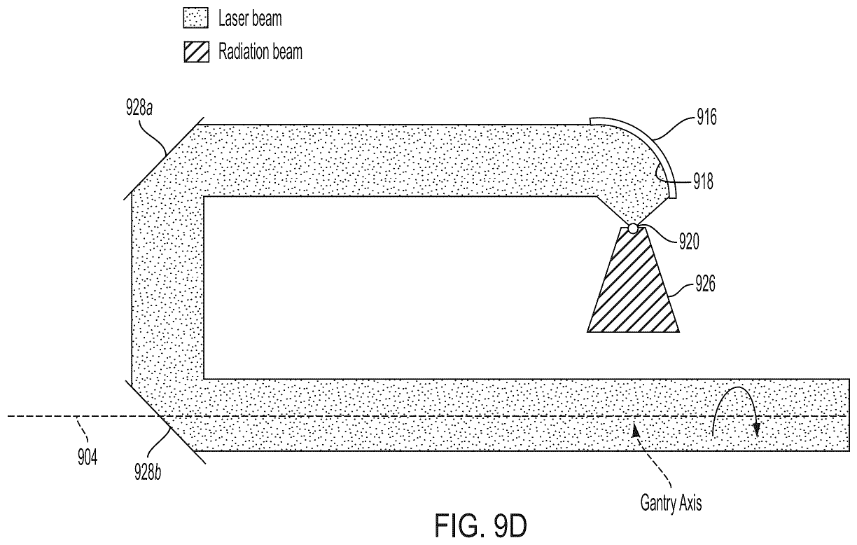

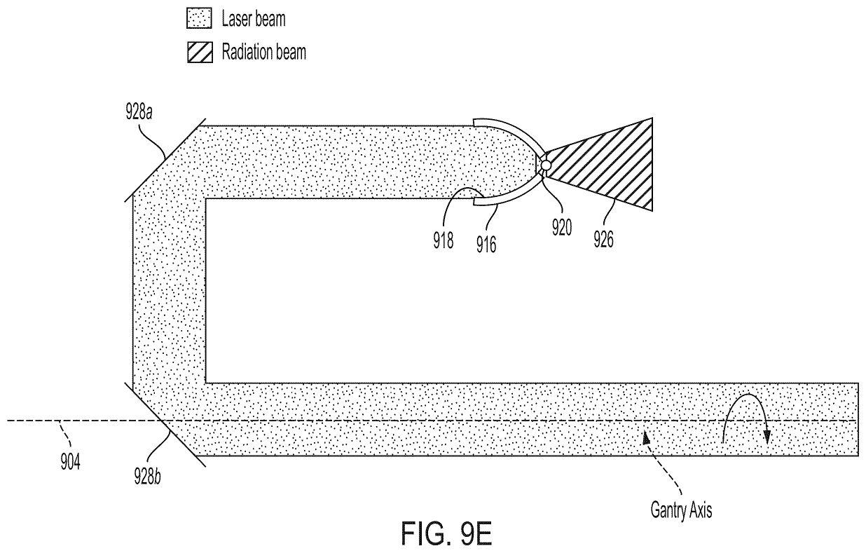

It will be appreciated that the plurality of gantry optical elements can be arranged in different optical configurations, as shown in the example optical configurations of FIGS. 9A-9C. Features common to both FIG. 8 and FIGS. 9A-9C are related via coordinated numerals that differ in increment by one hundred. In FIG. 9A, the example optical configuration includes gantry optical elements 928 (e.g., mirrors), one of which 928b, is disposed on the gantry axis 904 of the gantry. Moreover, the optical element 916 of the apparatus includes a high numerical aperture, on-axis parabolic mirror. As another example, FIG. 9B presents a schematic diagram of the example optical configuration of FIG. 9A, but in which the gantry optical element 928a includes an aperture for passing ionizing radiation therethrough. This latter configuration may be useful in applications where the beam of ionizing radiation 926 must minimally overlap (or not overlap) the path of the laser beam (e.g., the pulse of laser light).

FIG. 9C presents a schematic diagram of a further variation of the optical configuration of FIG. 9B. In FIG. 9C, the optical element 916 includes an aperture at the vertex of its parabolic shape. The aperture may allow for real-time dose monitoring of the beam of ionizing radiation 926. Such monitoring may also allow a control unit to continuously adjust an intensity of the laser beam (or pulses therein) to ensure that the beam of ionizing radiation 926 stays within a target intensity range (e.g., a constant intensity). In these variations, the beam of ionizing radiation 926 may include first and second portions that propagate in opposite directions from the focal point 920. The first portion may, for example, propagate along the collimator axis of the collimator, and the second portion may propagate through the aperture of the optical element 916 towards a radiation detector. In many instances, the first portion has an intensity that is greater than the second portion.

The optical element 916 may also be varied in configuration to control an orientation of the beam of ionizing radiation 926 relative to the laser beam, as shown in FIGS. 9D-9E. For example, FIG. 9D presents a schematic diagram of an example optical configuration for the gantry of FIG. 8, but in which the optical element 816 is a high numerical aperture, off axis parabolic mirror. Features common to both FIGS. 8 and 9D are related via coordinated numerals that differ in increment by one hundred. Here, the optical element 916 is used to orient the beam of ionizing radiation 926 perpendicular to that of the incoming laser beam. FIG. 9E presents a schematic diagram of the example optical configuration of FIG. 9D, but in which the optical element 916 is a high numerical aperture, parabolic transmission mirror. In this variation, the optical element 916 is used to orient the beam of ionizing radiation 926 such that the beam of ionizing radiation 926 propagates along a direction parallel to that of the incoming laser beam.

Although FIGS. 9A-9E are presented in the context of parabolic mirrors, and in particular, ones that have high numerical apertures, other types of optical elements are possible. FIG. 5 shows examples of other optical elements that are possible for the example system 800.

In some aspects of what is described, an apparatus may be described by the following examples. The apparatus may, in many cases, be used to generate ionizing radiation.
Example 1. An apparatus, comprising:
   a laser configured to generate a pulse of laser light;
   a gas medium comprising an interaction region therein; and
   an optical element comprising a reflective surface that defines a focal point in the interaction region, the reflective surface configured to receive the pulse of laser light and focus the pulse of laser light at the focal point;
   wherein the pulse of laser light is configured to generate a plasma in the interaction region when focused at the focal point by the reflective surface; and
   wherein the gas medium is configured to emit an ionizing radiation from the interaction region in response to the plasma being generated therein, the ionizing radiation comprising electron radiation.
Example 2. The apparatus of example 1, wherein the reflective surface is configured to focus the pulse of laser light at the focal point at or near the diffraction limit.
Example 3. The apparatus of example 1 or example 2, wherein the reflective surface is configured to focus the pulse of laser light at the focal point with a B-integral no greater than 2π radians.
Example 4. The apparatus of example 1 or any one of examples 2-3, wherein the pulse of laser light is configured to generate a relativistic ponderomotive force in the interaction region when focused onto the focal point.
Example 5. The apparatus of example 1 or any one of examples 2-4, wherein the ionizing radiation comprises a beam of ionizing radiation.
Example 6. The apparatus of example 1 or any one of examples 2-5, wherein the optical element has a numerical aperture of at least 0.25.
Example 7. The apparatus of example 1 or any one of examples 2-6, wherein the gas medium is air.
Example 8. The apparatus of example 1 or any one of examples 2-6, wherein the gas medium comprises a gaseous atom or molecule whose total electrons number greater than fourteen.
Example 9. The apparatus of example 1 or any one of examples 2-6, wherein the gas medium comprises a gaseous molecule whose total electrons number greater than fourteen, and the gaseous molecule comprises an atom whose atomic number is at least 35.
Example 10. The apparatus of example 1 or any one of examples 2-9, wherein the ionizing radiation comprises photon radiation.
Example 11. The apparatus of example 10, wherein the photon radiation comprises X-ray radiation.
Example 12. The apparatus of example 1 or any one of examples 2-11, comprising an electron-to-photon converter adjacent the interaction region.
Example 13. The apparatus of example 12,
   wherein the apparatus comprises a photon-to-neutron converter; and
   wherein the electron-to-photon converter resides between the interaction region and the photon-to-neutron converter.
Example 14. The apparatus of example 1 or any one of examples 2-13, wherein the pulse of laser light has a pulse duration no longer than 100 picoseconds.
Example 15. The apparatus of example 1 or any one of examples 2-14, wherein the pulse of laser light has a wavelength greater than 400 nm.
Example 16. The apparatus of example 1 or any one of examples 2-15, comprising:
   an optical pathway that extends between the laser and the optical element; and
   a second optical element disposed on the optical axis and comprising a deformable reflective surface and a transducer coupled thereto, the transducer configured to selectively deform the deformable reflective surface in response to receiving a signal that represents a target shape of the deformable reflective surface.
Example 17. The apparatus of example 1 or any one of examples 2-16, comprising:
   a gas cell containing the gas medium and the optical element, the gas cell comprising:
      a window transparent to the pulse of laser light and the ionizing radiation, the window disposed on a first side of the gas cell, and
      a mount on a second side of the gas cell opposite the first side, the mount coupled to the optical element and configured to selectively alter a position of the optical element relative to an optical axis of the gas cell;
   wherein the optical element is coupled to the mount such that the reflective surface faces the window.
Example 18. The apparatus of example 1 or any one of examples 2-16,
   wherein the reflective surface has a parabolic shape and comprises an aperture at a vertex of the parabolic shape;
   wherein the apparatus comprises a gas cell containing the gas medium and the optical element, the gas cell comprising:
      a first window transparent to the pulse of laser light and disposed on a first side of the gas cell,
      a second window transparent to the ionizing radiation and disposed on a second side of the gas cell opposite the first side, and
      a mount disposed between the first and second windows and coupled to the optical element, the mount configured to selectively alter a position of the optical element relative to an optical axis of the gas cell; and
   wherein the optical element is coupled to the mount such that the reflective surface faces the first window and the aperture faces the second window.

In some aspects of what is described, a method may be described by the following examples. The method may, in many cases, be used to generate ionizing radiation.
Example 19. A method, comprising:
   generating a pulse of laser light by operation of a laser;
   receiving the pulse of laser light at a reflective surface of an optical element, the reflective surface defining a focal point in an interaction region of a gas medium;
   focusing, by operation of the reflective surface, the pulse of laser light at the focal point to generate a plasma in the interaction region; and
   emitting an ionizing radiation from the interaction region in response to the plasma being generated therein, the ionizing radiation comprising electron radiation.
Example 20. The method of example 19, wherein the reflective surface is configured to focus the pulse of laser light at the focal point at or near the diffraction limit.
Example 21. The method of example 19 or example 20, wherein the reflective surface is configured to focus the pulse of laser light at the focal point with a B-integral no greater than 2π radians.
Example 22. The method of example 19 or any one of examples 20-21, wherein focusing the pulse of laser light comprises generating a relativistic ponderomotive force in the interaction region.
Example 23. The method of example 19 or any one of examples 20-22, wherein emitting an ionizing radiation comprises emitting a beam of ionizing radiation from the interaction region.
Example 24. The method of example 19 or any one of examples 20-23, wherein the optical element has a numerical aperture of at least 0.25.
Example 25. The method of example 19 or any one of examples 20-24, comprising:
   determining a target position of the reflective surface that corresponds to a maximum intensity of the ionizing radiation, the target position based on data that comprises measured intensities of the ionizing radiation and respective positions of the reflective surface; and
   securing the optical element to set the reflective surface in the target position.
Example 26. The method of example 25,
   wherein receiving the pulse of laser light comprises propagating the pulse of laser light along an optical axis that terminates at the reflective surface; and
   wherein determining the target position comprises:
      displacing the reflective surface to alter a position of the focal point relative to the optical axis, and
      measuring an intensity of the ionizing radiation emitted from the interaction region when the focal point is in the altered position.
Example 27. The method of example 19 or any one of examples 20-26, comprising:
   wherein receiving the pulse of laser light comprises propagating the pulse of laser light along an optical pathway that extends between the laser and the optical element;
   wherein a second optical element is disposed on the optical axis and comprises a deformable reflective surface and a transducer coupled thereto; and
   wherein the method comprises:
      receiving a signal at the second optical element that represents a target shape of the deformable reflective surface, and
      deforming, by operation of the transducer, the deformable reflective surface in response to the signal.
Example 28. The method of example 19 or any one of examples 20-27, wherein the gas medium is air.
Example 29. The method of example 19 or any one of examples 20-27, wherein the gas medium comprises a gaseous atom or molecule whose total electrons number greater than fourteen.
Example 30. The method of example 19 or any one of examples 20-27, wherein the gas medium comprises a gaseous molecule whose total electrons number greater than fourteen, and the gaseous molecule comprises an atom whose atomic number is at least 35.
Example 31. The method of example 19 or any one of examples 20-30, wherein the ionizing radiation comprises photon radiation.
Example 32. The method of example 31, wherein the photon radiation comprises X-ray radiation.
Example 33. The method of example 31 or example 32, comprising:
   converting, by operation of an electron-to-photon converter, at least a portion of the electron radiation to generate the photon radiation, the electron-to-photon converter disposed adjacent the interaction region.
Example 34. The method of example 33,
   wherein the electron-to-photon converter resides between the interaction region and a photon-to-neutron converter; and
   wherein the method comprises converting, by operation of the photon-to-neutron converter, at least a portion of the photon radiation to generate neutron radiation.
Example 35. The method of example 19 or any one of examples 20-34, wherein the pulse of laser light has a pulse duration no longer than 100 picoseconds.
Example 36. The method of example 19 or any one of examples 20-35, wherein the pulse of laser light has a wavelength greater than 400 nm.

In some aspects of what is described, a system may be described by the following examples. The system may, in many cases, be used to generate ionizing radiation.
Example 37. A system, comprising:
   a laser configured to generate a pulse of laser light;
   a gantry configured to rotate about a gantry axis and comprising:
      a collimator extending along a collimator axis between first and second collimator ends, the collimator axis intersecting the gantry axis at an isocenter, the first collimator end configured to face the isocenter while the gantry rotates about the gantry axis;
      an optical element proximate the second collimator end and comprising a reflective surface that defines a focal point in a gas medium, the reflective surface configured to receive the pulse of laser light and focus the pulse of laser light at the focal point, and
      the gas medium, adjacent the reflective surface and having an interaction region therein, the interaction region comprising the focal point,
      wherein:
         the pulse of laser light is configured to generate a plasma in the interaction region when focused at the focal point, and
         the gas medium is configured to emit a beam of ionizing radiation from the interaction region in response to the plasma being generated therein, the beam of ionizing radiation propagating towards the second collimator end and comprising a beam of electron radiation; and
   a support surface configured to support a target and position at least a portion thereof at the isocenter.
Example 38. The system of example 37, wherein the gantry comprises:
   a gantry housing; and
   a plurality of gantry optical elements internal to the gantry housing and configured to define at least part of an optical path between the laser and the optical element.
Example 39. The system of example 38, wherein the laser is disposed in the gantry housing and the optical path is internal to the gantry housing.
Example 40. The system of example 38,
   wherein the gantry housing comprises an optical port configured to receive the pulse of laser light from the laser; and
   wherein the optical path comprises a portion internal to the gantry housing, the portion extending between the optical port and the optical element.
Example 41. The system of example 37 or any one of examples 38-40, wherein the reflective surface is configured to focus the pulse of laser light at the focal point at or near the diffraction limit.
Example 42. The system of example 37 or any one of examples 38-41, wherein the reflective surface is configured to focus the pulse of laser light at the focal point with a B-integral no greater than 2π radians.
Example 43. The system of example 37 or any one of examples 38-42, wherein the pulse of laser light is configured to generate a relativistic ponderomotive force in the interaction region when focused onto the focal point.
Example 44. The system of example 37 or any one of examples 38-43, wherein the optical element has a numerical aperture of at least 0.25.
Example 45. The system of example 37 or any one of examples 38-44, wherein the gas medium is air.
Example 46. The system of example 37 or any one of examples 38-44, wherein the gas medium comprises a gaseous atom or molecule whose total electrons number greater than fourteen.
Example 47. The system of example 37 or any one of examples 38-44, wherein the gas medium comprises a gaseous molecule whose total electrons number greater than fourteen, and the gaseous molecule comprises an atom whose atomic number is at least 35.
Example 48. The system of example 37 or any one of examples 38-47, wherein the ionizing radiation comprises photon radiation.
Example 49. The system of example 48, wherein the photon radiation comprises X-ray radiation.
Example 50. The system of example 37 or any one of examples 38-49, comprising an electron-to-photon converter adjacent the interaction region.
Example 51. The system of example 50,
   wherein the apparatus comprises a photon-to-neutron converter; and
   wherein the electron-to-photon converter resides between the interaction region and the photon-to-neutron converter.
Example 52. The system of example 37 or any one of examples 38-51, wherein the pulse of laser light has a pulse duration no longer than 100 picoseconds.
Example 53. The system of example 37 or any one of examples 38-52, wherein the pulse of laser light has a wavelength greater than 400 nm.
Example 54. The system of example 37 or any one of examples 38-53, comprising:
   an optical pathway that extends between the laser and the optical element; and
   a second optical element disposed on the optical axis and comprising a deformable reflective surface and a transducer coupled thereto, the transducer configured to selectively deform the deformable reflective surface in response to receiving a signal that represents a target shape of the deformable reflective surface.

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple embodiments separately or in any suitable sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of embodiments have been described. Nevertheless, it will be understood that various modifications can be made. Accordingly, other embodiments are within the scope of the following claims.

Before examination, please amend the claims as follows:

1. An apparatus, comprising:
a laser configured to generate a pulse of laser light;
a gas medium comprising an interaction region therein; and
an optical element comprising a reflective surface that defines a focal point in the interaction region, the reflective surface configured to receive the pulse of laser light and focus the pulse of laser light at the focal point;
wherein the pulse of laser light is configured to generate a plasma in the interaction region when focused at the focal point by the reflective surface; and
wherein the gas medium is configured to emit an ionizing radiation from the interaction region in response to the plasma being generated therein, the ionizing radiation comprising electron radiation.

2. The apparatus of claim 1, wherein the reflective surface is configured to focus the pulse of laser light at the focal point at or near the diffraction limit.

3. The apparatus of claim 1, wherein the reflective surface is configured to focus the pulse of laser light at the focal point with a B-integral no greater than 2π radians.

4. The apparatus of claim 1, wherein the pulse of laser light is configured to generate a relativistic ponderomotive force in the interaction region when focused onto the focal point.

5. The apparatus of claim 1, wherein the ionizing radiation comprises a beam of ionizing radiation.

6. (canceled)

7. The apparatus of claim 1, wherein the gas medium is air.

8. The apparatus of claim 1, wherein the gas medium comprises a gaseous atom or molecule whose total electrons number greater than fourteen.

9. The apparatus of claim 1, wherein the gas medium comprises a gaseous molecule whose total electrons number greater than fourteen, and the gaseous molecule comprises an atom whose atomic number is at least 35.

10. The apparatus of claim 1, wherein the ionizing radiation comprises photon radiation.

11. The apparatus of claim 10, wherein the photon radiation comprises X-ray radiation or γ-ray radiation.

12. The apparatus of claim 1, comprising an electron-to-photon converter adjacent the interaction region.

13. The apparatus of claim 12,
wherein the apparatus comprises a photon-to-neutron converter; and
wherein the electron-to-photon converter resides between the interaction region and the photon-to-neutron converter.

14.-15. (canceled)

16. The apparatus of claim 1, comprising:
an optical pathway that extends between the laser and the optical element; and
a second optical element disposed on the optical axis and comprising a deformable reflective surface and a transducer coupled thereto, the transducer configured to selectively deform the deformable reflective surface in response to receiving a signal that represents a target shape of the deformable reflective surface.

17. The apparatus of claim 1, comprising:
a gas cell containing the gas medium and the optical element, the gas cell comprising:
a window transparent to the pulse of laser light and the ionizing radiation, the window disposed on a first side of the gas cell, and
a mount on a second side of the gas cell opposite the first side, the mount coupled to the optical element and configured to selectively alter a position of the optical element relative to an optical axis of the gas cell;
wherein the optical element is coupled to the mount such that the reflective surface faces the window.

18. (canceled)

19. A method, comprising:
generating a pulse of laser light by operation of a laser;
receiving the pulse of laser light at a reflective surface of an optical element, the reflective surface defining a focal point in an interaction region of a gas medium;
focusing, by operation of the reflective surface, the pulse of laser light at the focal point to generate a plasma in the interaction region; and
emitting an ionizing radiation from the interaction region in response to the plasma being generated therein, the ionizing radiation comprising electron radiation.

20. The method of claim 19, wherein the reflective surface is configured to focus the pulse of laser light at the focal point at or near the diffraction limit.

21. The method of claim 19, wherein the reflective surface is configured to focus the pulse of laser light at the focal point with a B-integral no greater than 2π radians.

22. The method of claim 19, wherein focusing the pulse of laser light comprises generating a relativistic ponderomotive force in the interaction region.

23. The method of claim 19, wherein emitting an ionizing radiation comprises emitting a beam of ionizing radiation from the interaction region.

24.-26. (canceled)

27. The method of claim 19, comprising:
wherein receiving the pulse of laser light comprises propagating the pulse of laser light along an optical pathway that extends between the laser and the optical element;
wherein a second optical element is disposed on the optical axis and comprises a deformable reflective surface and a transducer coupled thereto; and
wherein the method comprises:
receiving a signal at the second optical element that represents a target shape of the deformable reflective surface, and
deforming, by operation of the transducer, the deformable reflective surface in response to the signal.

28. The method of claim 19, wherein the gas medium is air.

29. The method of claim 19, wherein the gas medium comprises a gaseous atom or molecule whose total electrons number greater than fourteen.

30. The method of claim 19, wherein the gas medium comprises a gaseous molecule whose total electrons number greater than fourteen, and the gaseous molecule comprises an atom whose atomic number is at least 35.

31. The method of claim 19, wherein the ionizing radiation comprises photon radiation.

32. The method of claim 31, wherein the photon radiation comprises X-ray radiation or γ-ray radiation.

33. The method of claim 31, comprising:
converting, by operation of an electron-to-photon converter, at least a portion of the electron radiation to generate the photon radiation, the electron-to-photon converter disposed adjacent the interaction region.

34. The method of claim 33,
wherein the electron-to-photon converter resides between the interaction region and a photon-to-neutron converter; and
wherein the method comprises converting, by operation of the photon-to-neutron converter, at least a portion of the photon radiation to generate neutron radiation.

35.-54. (canceled)
